SEQUENCE LISTING (9 sequences)

SEQ ID NO: 1 - RNA, 21 nt, Artificial Sequence
  Description of Artificial Sequence mir-140 target sequence
agugguuuuacccuaugguag

SEQ ID NO: 2 - DNA, 41 bp, Artificial Sequence
  Description of Artificial Sequence probe for mir-140 target sequence
ctatgguccacuaccauaggguaaaaccacugctggtttta

SEQ ID NO: 3 - DNA, 38 bp, Artificial Sequence
  Description of Artificial Sequence probe for target sequence
uggattucacucaaatccagaggcuagcagacctagcc

SEQ ID NO: 4 - DNA, 38 bp, Artificial Sequence
  Description of Artificial Sequence probe for target sequence
tggatttcactcaaatccagaggctagcagacctagcc

SEQ ID NO: 5 - DNA, 38 bp, Artificial Sequence
  Description of Artificial Sequence probe for target sequence
tggatttcacucaaatccagaggcuagcagacctagcc

SEQ ID NO: 6 - RNA, 20 nt, Artificial Sequence
  Description of Artificial Sequence probe for siRNA molecule for PTEN
uuugucucugguccuuacuu

SEQ ID NO: 7 - DNA, 20 bp, Artificial Sequence
  Description of Artificial Sequence probe for siRNA molecule for PTEN
tttgtctctggtccttactt

SEQ ID NO: 8 - RNA, 20 nt, Artificial Sequence
  Description of Artificial Sequence probe for siRNA molecule for PTEN
aaguaaggaccagagacaaa

SEQ ID NO: 9 - DNA, 20 bp, Artificial Sequence
  Description of Artificial Sequence probe for siRNA molecule for PTEN
aagtaaggaccagagacaaa

(19) United States
(12) Patent Application Publication
(10) Pub. No.: US 2005/0064471 A1
(43) Pub. Date: Mar. 24, 2005

(54) Probe design for the detection of RNAs in cell extracts, cells, and tissues

(76) Inventors: Richard H. Griffey, Vista, CA (US); Lisa J. Marcus-Wanders, Carlsbad, CA (US)

Correspondence Address: COZEN O'CONNOR, P.C., 1900 MARKET STREET, PHILADELPHIA, PA, 19103-3508, US

(21) Appl. No.: 10/895,562

(22) Filed: Jul. 20, 2004

Related U.S. Application Data
(60) Provisional application No. 60/490,137, filed on Jul. 25, 2003.

(51) Int. Cl. C12Q 1/68; C07H 21/04

(52) U.S. Cl. 435/6; 536/24.3; 536/25.32

(57) ABSTRACT

The present invention provides probes comprising a bis-stem-loop structure, and to methods of detecting nucleic acid polymers using the same.

### CROSS-REFERENCE TO RELATED APPLICATIONS

This application claims priority to U.S. provisional application Ser. No. 60/490,137 filed Jul. 25, 2003, which is incorporated herein by reference in its entirety.

### FIELD OF THE INVENTION

The present invention is directed, in part, to nucleic acid probes comprising a bis-stem-loop structure, and to methods of detecting nucleic acid polymers using the same.

### BACKGROUND OF THE INVENTION

Specific detection of small natural RNA molecules such as small interfering RNA molecules (siRNA), micro RNA molecules (miRNA), or exogenously administered antisense RNA molecules is not straightforward. The abundance of 20 to 50 nucleotide RNA molecules in a typical eukaryotic cell can be quite high. Extraction-based approaches have no selectivity and often times have low efficiencies. Methods such as laser-induced fluorescence capillary electrophoresis, such as described in U.S. Pat. No. 6,045,995, can be used to detect RNA molecules from cell or tissue extracts. In many cases, however, it would also be desirable to ascertain the location of the RNA within the cell. Current methods, however, require microinjection of the fluorescent probe into the cells, which limits throughput and quantitation (see for example, Pederson, Nuc. Acids Res., 2001, 29, 1013-1016; Dirks et al., Methods, 2003, 29 51-57; and Perlette et al., Anal. Chem., 2001, 73, 5544-5550). Thus, development of new probes and detection systems for small nucleic acids is greatly needed.

### SUMMARY OF THE INVENTION

The present invention provides compounds comprising the structure R¹—Uₙ-Lₖ-Wₐ—Xᵣ—Yₚ-Eₘ-Z_{b}-R² that can be sued to detect and/or quantify target nucleic acid polymers. In the aforementioned structure, one of R¹ and R² is a fluorescent reporter group and the other of R¹ and R² is a quenching agent or second fluorescent reporter that can undergo fluorescence resonance energy transfer (FRET) with R¹. Each U and each W are nucleobases, wherein at least one U nucleobase is complementary to a corresponding W nucleobase. Each Y and each Z are nucleobases, wherein at least one Y nucleobase is complementary to a corresponding Z nucleobase. Each L and each E is a nucleobase that, when a plurality of U and a plurality of W nucleobases are basepaired and a plurality of Y and a plurality of Z nucleobases are basepaired, forms a loop. Each X is a nucleobase that, when a plurality of U and a plurality of W nucleobases are basepaired and a plurality of Y and a plurality of Z nucleobases are basepaired, is not basepaired to any nucleobase of the compound. The variables n, a, p, and b are, independently, 4 to 10; the variables k and m are, independently, 1 to 10; and the variable r is 0 to 4. In addition, groups such as alkyl carboxylic acids or alkyl thiols can be employed to link R¹ and/or R² to the oligonucleotide.

The present invention also provides methods of detecting a target nucleic acid polymer by detecting the presence of a fluorescent signal produced by a probe compound of the invention when contacted with the target nucleic acid polymer. The target nucleic acid polymer can be an RNA molecule such as, for example, a small interfering RNA, mRNA, antisense RNA, or micro RNA.

### DESCRIPTION OF EMBODIMENTS

The present invention provides compounds that can function as probes for the detection and/or quantifying of nucleobase polymers. The compounds of the invention can be used to detect the presence and concentration of small and large RNA molecules, for example, in cells and tissues. The compounds of the invention utilize the formation of specific hydrogen bonds between base pairs to generate specificity of binding. The compounds of the invention comprise the structure R¹—Uₙ-Lₖ-Wₐ—Xᵣ—Yₚ-Eₘ-Z_{b}-R².

One of R¹ and R² is a fluorescent reporter group and the other of R¹ and R² is a quenching agent. Thus, in one embodiment, R¹ is a fluorescent reporter group and R² is a quenching agent. Alternately, in another embodiment, R² is a fluorescent reporter group and R¹ is a quenching agent. In some embodiments, the fluorescent reporter group is a fluorescent base. In some embodiments, the fluorescent reporter group is a fluorophore including, but not limited to, tetramethylrhodamine (TAMRA), fluorescein, Cy3, Cy3.5, Cy5, Cy5.5, Texas Red, and the like. Fluorescent bases and fluorophores, as well as additional fluorescent reporter groups, are well known to those skilled in the art and can be found in, for example, Handbook Of Fluorescent Probes and research Chemicals, 9^{th} Ed., Molecular Probes, Inc. In some embodiments, the quenching agent is an arylazo compound including, but not limited to, 4-(4′-dimethylaminophenylazo)benzoic acid, a Black Hole Quencher™ (Biosearch Technologies, Inc., Novato, Calif.) or Iowa Black™ (Integrated DNA Technologies, Inc., Coralville, Iowa.), and the like. Additional quenching agents are also well known to those skilled in the art. In addition, groups such as alkyl carboxylic acids or alkyl thiols can be employed to link R¹ and/or R² to the oligonucleotide.

In other embodiments, both R¹ and R² are fluorescent reporter groups. In such embodiments, these fluorescent reporter groups can undergo fluorescence resonance energy transfer (FRET).

Each U and each W variable in the above structure represents a nucleobase. Referring to the above structure, n and a, independently, are 4 to 10 (e.g., 4, 5, 6, 7, 8, 9, or 10, or any range therein). Thus, there can be from 4 to 10 U nucleobases and from 4 to 10 W nucleobases in any compound. In some embodiments, there can be from 5 to 7 U nucleobases and from 5 to 7 W nucleobases in any compound. In addition, the number of U nucleobases in a particular compound can be the same as the number of W nucleobases in the same compound or can be different than the number of W nucleobases in the same compound. Each L variable in the above structure also represents a nucleobase. Referring to the above structure, k is 1 to 10 (e.g., 1, 2, 3, 4, 5, 6, 7, 8, 9, or 10, or any range therein). Thus, there can be from 1 to 10 L nucleobases in any compound. In some embodiments, there can be from 3 to 8 L nucleobases in any compound.

The Uₙ-Lₖ-Wₐ portion of the above structure forms a 5′ stem-loop structure within any of the compounds of the invention. At least one U nucleobase within the 5′stem-loop is complementary to (and capable of basepairing with) at least one corresponding W nucleobase. A corresponding nucleobase is that nucleobase that is positioned to be basepaired when a stem-loop structure is formed. For example, the 5′-most U nucleobase can basepair with the 3′-most W nucleobase, and the 3′-most U nucleobase can basepair with the 5′-most W nucleobase. In some embodiments, the number of U nucleobases and the number of W nucleobases is the same, and each U nucleobase is complementary to each corresponding W nucleobase. In other embodiments, although the number of U nucleobases and the number of W nucleobases is the same, there can be a mismatch between corresponding U and W nucleobases. Alternately, the number of U nucleobases and the number of W nucleobases may not be the same, in which case there will be a bulge within the set of U nucleobases or the set of W nucleobases. Each L nucleobase forms a portion of the loop within the 5′stem-loop structure. The set of L nucleobases, thus, forms the loop of the 5′ stem-loop structure.

Each Y and each Z variable in the above structure also represents a nucleobase. Referring to the above structure, p and b, independently, are 4 to 10 (e.g., 4, 5, 6, 7, 8, 9, or 10, or any range therein). Thus, there can be from 4 to 10 Y nucleobases and from 4 to 10 Z nucleobases in any compound. In some embodiments, there can be from 5 to 7 Y nucleobases and from 5 to 7 Z nucleobases in any compound. In addition, the number of Y nucleobases in a particular compound can be the same as the number of Z nucleobases in the same compound or can be different than the number of Z nucleobases in the same compound. Each E variable in the above structure also represents a nucleobase. Referring to the above structure, m is 1 to 10 (e.g., 1, 2, 3, 4, 5, 6, 7, 8, 9, or 10, or any range therein). Thus, there can be from 1 to 10 E nucleobases in any compound. In some embodiments, there can be from 3 to 8 E nucleobases in any compound.

The Yₚ-Eₘ-Z_{b} portion of the above structure also forms a 3′ stem-loop structure within any of the compounds of the invention. At least one Y nucleobase within the 3′ stem-loop is complementary to (and capable of basepairing with) at least one corresponding Z nucleobase. A corresponding nucleobase is that nucleobase that is positioned to be basepaired when a stem-loop structure is formed. For example, the 5′-most Y nucleobase can basepair with the 3′-most Z nucleobase, and the 3′-most Y nucleobase can basepair with the 5′-most Z nucleobase. In some embodiments, the number of Y nucleobases and the number of Z nucleobases is the same, and each Y nucleobase is complementary to each corresponding Z nucleobase. In other embodiments, although the number of Y nucleobases and the number of Z nucleobases is the same, there can be a mismatch between corresponding Y and Z nucleobases. Alternately, the number of Y nucleobases and the number of Z nucleobases may not be the same, in which case there will be a bulge within the set of Y nucleobases or the set of Z nucleobases. Each E nucleobase forms a portion of the loop within the 3′ stem-loop structure. The set of E nucleobases, thus, forms the loop of the 3′ stem-loop structure.

The 5′ stem-loop and the 3′ stem-loop described above can have different numbers of nucleobases comprising the stem portions of each stem-loop. For example, the 5′ stem-loop can have 12 nucleobases within the stem portion (i.e., the sum of all U and W nucleobases is 12), whereas the 3′ stem-loop can have 14 nucleobases within the stem portion (i.e., the sum of all Y and Z nucleobases is 14). Thus, the 5′ stem-loop and the 3′ stem-loop need not be symmetrical in regard to the number of nucleobases within the stems. Similarly, the 5′ stem-loop and the 3′ stem-loop described above can have different numbers of nucleobases comprising the loop portions of each stem-loop. For example, the 5′ stem-loop can have 7 nucleobases within the loop portion (i.e., 7 L nucleobases), whereas the 3′ stem-loop can have 8 nucleobases within the loop portion (i.e., 8 E nucleobases).

The 5′ stem-loop and the 3′ stem-loop described above are linked together at the 3′-most W nucleobase and the 5′-most Y nucleobase by the X variable depicted in the structure shown above. The X variable is also a nucleobase. Referring to the above structure, r is 0 to 4 (i.e., 0, 1, 2, 3, or 4, or any range therein). In some embodiments, r is 1 or 2. Alternately, the X nucleobase can be absent from a compound.

The entire structure containing both the 5′ stem-loop structure and the 3′ stem-loop structure linked by the X nucleobases, thus, forms a bis-stem-loop structure. This structure provides nuclease resistance at the 3′ and 5′ ends with the need for incorporating phosphorothioate backbone modifications and should reduce non-specific interactions with single-strand binding proteins in cells and tissues. The bis-stem-loop structure is resistant to the activity of cellular helicases, which are enzymes that unwind nucleic acid duplexes. In contrast to the present invention, conventional molecular beacons with a single short stem can be unwound by helicases and produce background fluorescence that can not be differentiated from signal produced by hybridization to the desired target RNA. In addition, binding to proteins should not open the bis-stem-loop structure, which can occur with conventional beacons when single strand binding proteins attach to the large single stranded loop.

The bis-stem-loop structure can be adjusted to compensate for differences in the thermal stabilities of the two stems. For example, the number of base pairs in one stem can be increased from 7 to 8 when the stem is AT-rich. In G-C rich stems, the number of base pairs can be decreased from 7 to 6, for example, or the loop size could be increased, or both. Alternative, chemical modifications can be introduced where desired to increase thermodynamic stability. In addition, if so desired, the stabilities of the two stems can be made to be equivalent. In some embodiments, at least one W, X, or Y nucleobase comprises a chemical modification such as, for example, 2′-O-methyl, 2′-O-methoxyethyl, or 2′F. These modifications can be introduced to enhance the hybridization affinity for RNA. In some embodiments, at least one U or Z nucleobase comprises a chemical modification such as, for example, a 2′-deoxy or 2′-arafluoro. In some embodiments, a compound of the invention will comprise less than five contiguous deoxyribonucleotides, which helps to avoid RNAse H-mediated cleavage of undesired targets in the cell.

The compounds of the invention can be used as probes to detect the presence and/or concentration of nucleic acid polymers. Nucleic acid polymers include, but are not limited to, siRNA, miRNA, mRNA, antisense oligonucleotides (DNA or RNA), and the like. The compounds of the invention can be used to detect the presence and/or concentration of nucleic acid polymers having between about 10 nucleotides and about 100 nucleotides, having between about 15 nucleotides and about 75 nucleotides, having between about 25 nucleotides and about 75 nucleotides, having between about 15 nucleotides and about 25 nucleotides, having between about 15 nucleotides and about 50 nucleotides, and having between about 25 nucleotides and about 50 nucleotides. A target nucleic acid polymer is a nucleic acid polymer which is desired to be detected or quantified. The target nucleic acid polymer can be detected in cell extracts, within cells, or within tissues or tissue sections.

The compounds of the invention comprise a target binding region for hybridizing to appropriate target nucleic acid polymers. The target binding region of a compound comprises all or some of the W, X, and Y nucleobases. In some embodiments, the target binding region comprises all of the W, X, and Y nucleobases. In some embodiments, the target binding region also comprises some or all of the L and/or E nucleobases present in the loops of the 5′ stem-loop structure and 3′ stem-loop structure, respectively. Thus, together, the W, X, and Y nucleobases comprise a nucleotide sequence, along with some or all of the L and/or E nucleobases, that is complementary to the nucleotide sequence of a target nucleic acid polymer. As discussed below, the target binding region of the probe compound need not be 100% complementary to the nucleotide sequence of the target nucleic acid polymer.

The bis-stem-loop structure allows for the ability to bring together in spatial proximity the quenching agent on either the 5′ or 3′ end with the fluorescent reporter group on the other of the 3′ or 5′ end. The X nucleobases form a gap that provides a space for the fluorescent reporter group and the quenching agent to stack onto the ends of the helical stems. In the absence of a target nucleic acid polymer, the bis-stem-loop structure of a compound of the invention retains its configuration. As a result, any fluorescence activity of the fluorescence reporter group is quenched by the quenching agent. In contrast, in the presence of an appropriate target nucleic acid molecule, the target binding region of the probe compound hybridizes to the target nucleic acid molecule, thus opening the bis-stem-loop structure of the probe compound. As a result, the quenching agent is no longer in spatial proximity to the fluorescence reporter group, and fluorescence emission can be detected.

“Complementary,” as used herein, refers to the capacity for precise pairing between two nucleobases of a probe compound. For example, if a nucleobase at a certain position of a probe compound is capable of hydrogen bonding with a nucleobase at a certain other position of the probe compound, then the position of hydrogen bonding between the two nucleobases of the probe compound is considered to be a complementary position. “Complementary,” as used herein, can also refer to the capacity for precise pairing between a nucleobases of a probe compound and a target nucleic acid polymer. The probe compound and the further DNA, RNA, or oligonucleotide target nucleic acid polymer are complementary to each other when a sufficient number of complementary positions in each molecule are occupied by nucleobases which can hydrogen bond with each other. Thus, “complementary” can be used to indicate a sufficient degree of precise pairing or complementarity over a sufficient number of nucleobases such that stable and specific binding occurs between the probe compound and a target nucleic acid polymer.

It is understood in the art that the nucleobase sequence of a probe compound need not be 100% complementary to that of its target nucleic acid polymer. Moreover, a probe compound may hybridize over one or more segments such that intervening or adjacent segments are not involved in the hybridization event (e.g., a loop structure or hairpin structure). The probe compounds of the present invention can comprise at least 70%, at least 75%, at least 80%, at least 85%, at least 90%, at least 95%, at least 96%, at least 97%, at least 98%, at least 99%, or 100% sequence complementarity to a target region within a target nucleic acid polymer. For example, a probe compound in which 18 of 20 nucleobases of the compound are complementary to a target region, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, a probe compound having a target binding region that is 18 nucleobases in length having 4 (four) noncomplementary nucleobases which are flanked by two regions of complete complementarity with the target nucleic acid polymer would have 77.8% overall complementarity with the target nucleic acid polymer and would thus fall within the scope of the present invention. Percent complementarity of a target binding region of a probe compound with a region of a target nucleic acid polymer can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656). Percent complementarity can also be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489).

The present invention also provides methods of detecting a target nucleic acid polymer by detecting the presence of a fluorescent signal produced by a probe compound of the invention when contacted with the target nucleic acid polymer. In some embodiments, a cell, cell extract, or tissue that may contain a target nucleic acid polymer whose detection is sought, is contacted with a probe compound of the invention. In some embodiments, the cell, cell extract, or tissue may be present within a container such as a microtiter plate, a test tube, or on a slide.

For target nucleic acid polymers within cells, a cell extract can be obtained by lysing the cell in a hypotonic buffer. The cellular debris can be removed by centrifugation. The probe compound can be added to the supernatant, and the entire mixture can be loaded into a capillary electrophoresis column, for example, and run in a non-denaturing mode. The fluorescence signals generated by the duplex formed between the probe compound and the complementary target nucleic acid polymer can be detected using laser-induced fluorescence excitation and detection. Non-specific complexes between the probe compound and any protein or between the probe compound and other nucleic acid molecules can be differentiated based on their electrophoretic mobility. The observed fluorescent intensity can be normalized to total RNA content in cells using conventional methods such as ribogreen fluorescence or RT-PCR analysis of a control mRNA such as GAPDH.

For target nucleic acid polymers within intact cells or tissues, the target nucleic acid polymer can be detected following transfection or electroporation of the probe compound into the cell(s) or tissue(s) of interest. The appearance of the fluorescent signal can be monitored as a function of probe concentration, temporally (Perlette, et al., Anal. Chem., 2001, 73, 5544-5550), or both to establish the level of hybridization to the target nucleic acid polymer by using, for example, flow cytometry or microscopy, such as confocal microscopy. Comparison of this level to a standard or standard curve can be performed to quantify the abundance of the target nucleic acid polymer in the cells. Alternately, a probe compound can be washed over a fixed tissue section on a slide. Following a series of stringency washes, the location of the induced fluorescence can be measured using microscopy.

The present invention also provides kits comprising any one or more of the probe compounds of the invention. The kit can also include cell lysis reagents, buffers, microscope slides, microtiter plates, and the like.

The nucleobases can be natural nucleobases or modified nucleobases. As used herein, “unmodified” or “natural” nucleobases include, but are not limited to, the purine bases adenine (A) and guanine (G), and the pyrimidine bases thymine (T), cytosine (C) and uracil (U). Modified nucleobases include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Additional modified nucleobases include, but are not limited to, tricyclic pyrimidines such as phenoxazine cytidine(1H-pyrimido(5,4-b)(1,4)benzoxazin-2(3H)-one), phenothiazine cytidine(1H-pyrimido(5,4-b)(1,4)benzothiazin-2(3H)-one), G-clamps such as a substituted phenoxazine cytidine (e.g. 9-(2-aminoethoxy)-H-pyrimido(5,4-b)(1,4)benzoxazin-2(3H)-one), carbazole cytidine(2H-pyrimido(4,5-b)indol-2-one), pyridoindole cytidine(H-pyrido(3′,2′:4,5)pyrrolo(2,3-d)pyrimidin-2-one). Modified nucleobases can also include those in which the purine or pyrimidine base is replaced with other heterocycles, for example 7-deaza-adenine, 7-deazaguanosine, 2-aminopyridine and 2-pyridone. Additional nucleobases include, but are not limited to, those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Some of these nucleobases are particularly useful for increasing the binding affinity of the compounds of the invention. These include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. and are presently suitable base substitutions, even more particularly when combined with 2′-O-methoxyethyl sugar modifications.

Representative U.S. patents that teach the preparation of certain of the above noted modified nucleobases as well as other modified nucleobases include, but are not limited to, the above noted U.S. Pat. Nos. 3,687,808; 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,681,941; and 5,750,692.

The compounds of the present invention can also have additional modifications. As is known in the art, a nucleoside is a base-sugar combination. The base portion of the nucleoside is normally a heterocyclic base. The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming probe compounds, the phosphate groups covalently link adjacent nucleosides to one another to form a linear polymeric compound. Within probe compounds, the phosphate groups are commonly referred to as forming the intemucleoside backbone of the compound. The normal linkage or backbone of RNA and DNA is a 3′ to 5′ phosphodiester linkage.

Specific examples of modifications useful in this invention include modified backbones or non-natural intemucleoside linkages. As defined in this specification, compounds having modified backbones include those that retain a phosphorus atom in the backbone and those that do not have a phosphorus atom in the backbone. For the purposes of this specification, and as sometimes referenced in the art, modified compounds that do not have a phosphorus atom in their intemucleoside backbone can also be considered to be probe compounds.

Suitable modified backbones containing a phosphorus atom therein include, for example, phosphorothioates, chiral phosphorothioates, phosphorodithioates, phosphotriesters, aminoalkylphosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, selenophosphates and boranophosphates having normal 3′-5′ linkages, 2′-5′ linked analogs of these, and those having inverted polarity wherein one or more internucleotide linkages is a 3′ to 3′, 5′to 5′, or 2′ to 2′ linkage. Compounds having inverted polarity comprise a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included.

Representative U.S. patents that teach the preparation of the above phosphorus-containing linkages include, but are not limited to: U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697; and 5,625,050.

Suitable modified backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative U.S. patents that teach the preparation of the above linkages include, but are not limited to: U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439.

In other embodiments, mimetics, both the sugar and the internucleoside linkage (i.e. the backbone), of the nucleotide units are replaced with novel groups. The nucleobase units are maintained for hybridization with an appropriate target nucleic acid polymer. One such compound, a mimetic that has been shown to have excellent hybridization properties, is referred to as a peptide nucleic acid (PNA). In PNA compounds, the sugar-backbone is replaced with an amide containing backbone, in particular an aminoethylglycine backbone. The nucleobases are retained and are bound directly or indirectly to aza nitrogen atoms of the amide portion of the backbone. Representative U.S. patents that teach the preparation of PNA compounds include, but are not limited to, U.S. Pat. Nos. 5,539,082, 5,714,331, and 5,719,262. Further teaching of PNA compounds can be found in Nielsen et al., Science, 1991, 254, 1497-1500.

Some embodiments of the invention are compounds with phosphorothioate backbones and compounds with heteroatom backbones, and in particular —CH₂—NH—O—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester backbone is represented as —O—P—O—CH₂—) of the above referenced U.S. Pat. No. 5,489,677, and the amide backbones of the above referenced U.S. Pat. No. 5,602,240. Also included are compounds having morpholino backbone structures of the above-referenced U.S. Pat. No. 5,034,506.

Modified compounds may also contain one or more substituted sugar moieties. In some embodiments, compounds can comprise one of the following at the 2′ position: OH; F; O—, S—, or N-alkyl; O—, S—, or N-alkenyl; O—, S— or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. In other embodiments, a compound can include O((CH₂)ₙO)ₘCH₃, O(CH₂)ₙOCH₃, O(CH₂)ₙNH₂, O(CH₂)ₙCH₃, O(CH₂)ₙONH₂, and O(CH₂)ₙON((CH₂)ₙCH₃)₂, where n and m are from 1 to about 10. Other compounds can comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, poly-alkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. In some embodiments, the modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. A further modification includes 2′-dimethylaminooxyethoxy, i.e., a O(CH₂)₂ON(CH₃)₂ group, also known as 2′-DMAOE and 2′-dimethylaminoethoxyethoxy (also known in the art as 2′-O-dimethyl-amino-ethoxy-ethyl or 2′-DMAEOE), i.e., 2′-O—CH₂—O—CH₂—N(CH₃)₂.

Other modifications include 2′-methoxy(2′-O—CH₃), 2′-aminopropoxy(2′-OCH₂CH₂CH₂NH₂), 2′-allyl(2′-CH₂—CH═CH₂), 2′-O-allyl (2′-O—CH₂—CH═CH₂) and 2′-fluoro (2′-F). The 2′-modification may be in the arabino (up) position or ribo (down) position. A suitable 2′-arabino modification is 2′-F. Similar modifications may also be made at other positions on the compound, particularly the 3′ position of the sugar on the 3′ terminal nucleotide or in 2′-5′ linked oligonucleotides and the 5′ position of 5′ terminal nucleotide. Compounds may also have sugar mimetics such as cyclobutyl moieties in place of the pentofuranosyl sugar. Representative U.S. patents that teach the preparation of such modified sugar structures include, but are not limited to: U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; and 5,700,920.

A further modification of the sugar includes Locked Nucleic Acids (LNAS) in which the 2′-hydroxyl group is linked to the 3′ or 4′ carbon atom of the sugar ring, thereby forming a bicyclic sugar moiety. The linkage can be a methylene (—CH₂—)ₙ group bridging the 2′ oxygen atom and the 4′ carbon atom wherein n is 1 or 2. LNAs and preparation thereof are described in WO 98/39352 and WO 99/14226.

The probe compounds used in accordance with this invention can be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare probe compounds such as the phosphorothioates and alkylated derivatives.

Unsubstituted and substituted phosphodiester (P═O) compounds are synthesized on an automated DNA synthesizer (Applied Biosystems model 394) using standard phosphoramidite chemistry with oxidation by iodine.

Phosphorothioates (P═S) are synthesized similar to phosphodiester compounds with the following exceptions: thiation was effected by utilizing a 10% w/v solution of 3,H-1,2-benzodithiole-3-one 1,1-dioxide in acetonitrile for the oxidation of the phosphite linkages. The thiation reaction step time was increased to 180 seconds and preceded by the normal capping step. After cleavage from the CPG column and deblocking in concentrated ammonium hydroxide at 55° C. (12-16 hr), the compounds were recovered by precipitating with >3 volumes of ethanol from a 1 M NH₄OAc solution. Phosphinate compounds are prepared as described in U.S. Pat. No. 5,508,270. Alkyl phosphonate compounds are prepared as described in U.S. Pat. No. 4,469,863. 3′-deoxy-3′-methylene phosphonate compounds are prepared as described in U.S. Pat. Nos. 5,610,289 or 5,625,050. Phosphoramidite compounds are prepared as described in U.S. Pat. Nos. 5,256,775 or 5,366,878. Alkylphosphonothioate compounds are prepared as described in published PCT applications WO 94/17093 and WO 94/02499. 3′-deoxy-3′-amino phosphoramidate compounds are prepared as described in U.S. Pat. No. 5,476,925. Phosphotriester compounds are prepared as described in U.S. Pat. No. 5,023,243. Borano phosphate compounds are prepared as described in U.S. Pat. Nos. 5,130,302 and 5,177,198.

Methylenemethylimino linked compounds, also identified as MMI linked compounds, methylenedimethylhydrazo linked compounds, also identified as MDH linked compounds, and methylenecarbonylamino linked compounds, also identified as amide-3 linked compounds, and methyleneaminocarbonyl linked compounds, also identified as amide-4 linked compounds, as well as mixed backbone compounds having, for instance, alternating MMI and P═O or P═S linkages are prepared as described in U.S. Pat. Nos. 5,378,825, 5,386,023, 5,489,677, 5,602,240, and 5,610,289.

Formacetal and thioformacetal linked compounds are prepared as described in U.S. Pat. Nos. 5,264,562 and 5,264,564. Ethylene oxide linked compounds are prepared as described in U.S. Pat. No. 5,223,618.

In general, RNA synthesis chemistry is based on the selective incorporation of various protecting groups at strategic intermediary reactions. Although one of ordinary skill in the art will understand the use of protecting groups in organic synthesis, a useful class of protecting groups includes silyl ethers. In particular, bulky silyl ethers are used to protect the 5′-hydroxyl in combination with an acid-labile orthoester protecting group on the 2′-hydroxyl. This set of protecting groups is then used with standard solid-phase synthesis technology. It is important to lastly remove the acid labile orthoester protecting group after all other synthetic steps. Moreover, the early use of the silyl protecting groups during synthesis ensures facile removal when desired, without undesired deprotection of 2′ hydroxyl.

Following this procedure for the sequential protection of the 5′-hydroxyl in combination with protection of the 2′-hydroxyl by protecting groups that are differentially removed and are differentially chemically labile, RNA compounds are synthesized. RNA compounds are synthesized in a stepwise fashion. Each nucleotide is added sequentially (3′-to 5′-direction) to a solid support-bound compound. The first nucleoside at the 3′-end of the chain is covalently attached to a solid support. The nucleotide precursor, a ribonucleoside phosphoramidite, and activator are added, coupling the second base onto the 5′-end of the first nucleoside. The support is washed and any unreacted 5′-hydroxyl groups are capped with acetic anhydride to yield 5′-acetyl moieties. The linkage is then oxidized to the more stable and ultimately desired P(V) linkage. At the end of the nucleotide addition cycle, the 5′-silyl group is cleaved with fluoride. The cycle is repeated for each subsequent nucleotide.

Following synthesis, the methyl protecting groups on the phosphates are cleaved in 30 minutes utilizing 1 M disodium-2-carbamoyl-2-cyanoethylene-1,1-dithiolate trihydrate (S₂Na₂) in DMF. The deprotection solution is washed from the solid support-bound compounds using water. The support is then treated with 40% methylamine in water for 10 minutes at 55° C. This releases the RNA compounds into solution, deprotects the exocyclic amines, and modifies the 2′-groups. The compounds can be analyzed by anion exchange HPLC at this stage.

The 2′-orthoester groups are the last protecting groups to be removed. The ethylene glycol monoacetate orthoester protecting group developed by Dharmacon Research, Inc. (Lafayette, Colo.), is one example of a useful orthoester protecting group which, has the following important properties. It is stable to the conditions of nucleoside phosphoramidite synthesis and compound synthesis. However, after compound synthesis the compounds is treated with methylamine which not only cleaves the compounds from the solid support but also removes the acetyl groups from the orthoesters. The resulting 2-ethyl-hydroxyl substituents on the orthoester are less electron withdrawing than the acetylated precursor. As a result, the modified orthoester becomes more labile to acid-catalyzed hydrolysis. Specifically, the rate of cleavage is approximately 10 times faster after the acetyl groups are removed. Therefore, this orthoester possesses sufficient stability in order to be compatible with compound synthesis and yet, when subsequently modified, permits deprotection to be carried out under relatively mild aqueous conditions compatible with the final RNA compound product.

Additionally, methods of RNA synthesis are well known in the art (Scaringe, S. A. Ph.D. Thesis, University of Colorado, 1996; Scaringe, S. A., et al., J. Am. Chem. Soc., 1998, 120, 11820-11821; Matteucci, M. D. and Caruthers, M. H. J. Am. Chem. Soc., 1981, 103, 3185-3191; Beaucage, S. L. and Caruthers, M. H. Tetrahedron Lett., 1981, 22, 1859-1862; Dahl, B. J., et al., Acta Chem. Scand,. 1990, 44, 639-641; Reddy, M. P., et al., Tetrahedrom Lett., 1994, 25, 4311-4314; Wincott, F. et al., Nucleic Acids Res., 1995, 23, 2677-2684; Griffin, B. E., et al., Tetrahedron, 1967, 23, 2301-2313; Griffin, B. E., et al., Tetrahedron, 1967, 23, 2315-2331).

In some embodiments, compounds are synthesized via solid phase P(III) phosphoramidite chemistry on an automated synthesizer capable of assembling 96 sequences simultaneously in a 96-well format. Phosphodiester intemucleotide linkages are afforded by oxidation with aqueous iodine. Phosphorothioate intemucleotide linkages are generated by sulfurization utilizing 3,H-1,2 benzodithiole-3-one 1,1 dioxide (Beaucage Reagent) in anhydrous acetonitrile. Standard base-protected beta-cyanoethyl-diiso-propyl phosphoramidites can be purchased from commercial vendors (e.g. PE-Applied Biosystems, Foster City, Calif., or Pharmacia, Piscataway, N.J.). Non-standard nucleosides are synthesized as per standard or patented methods. They are utilized as base protected beta-cyanoethyldiisopropyl phosphoramidites.

Compounds can be cleaved from support and deprotected with concentrated NH₄OH at elevated temperature (55-60° C.) for 12-16 hours and the released product then dried in vacuo. The dried product is then re-suspended in sterile water to afford a master plate from which all analytical and test plate samples are then diluted utilizing robotic pipettors.

The concentration of compound in each well can be assessed by dilution of samples and UV absorption spectroscopy. The full-length integrity of the individual products can be evaluated by capillary electrophoresis (CE) in either the 96-well format (Beckman P/ACE™ MDQ) or, for individually prepared samples, on a commercial CE apparatus (e.g., Beckman P/ACE™ 5000, ABI 270). Base and backbone composition can be confirmed by mass analysis of the compounds utilizing electrospray-mass spectroscopy. All assay test plates can be diluted from the master plate using single and multi-channel robotic pipettors. Plates can be judged to be acceptable if at least 85% of the compounds on the plate were at least 85% full length.

Poly(A)+ mRNA can be isolated according to Miura et al., (Clin. Chem., 1996, 42, 1758-1764). Other methods for poly(A)+ mRNA isolation are routine in the art. Briefly, for cells grown on 96-well plates, growth medium is removed from the cells and each well is washed with 200 μL cold PBS. 60 μL lysis buffer (10 mM Tris-HCl, pH 7.6, 1 mM EDTA, 0.5 M NaCl, 0.5% NP-40, 20 mM vanadyl-ribonucleoside complex) is added to each well, the plate is gently agitated and then incubated at room temperature for five minutes. 55 μL of lysate is transferred to Oligo d(T) coated 96-well plates (AGCT Inc., Irvine Calif.). Plates are incubated for 60 minutes at room temperature, washed 3 times with 200 μL of wash buffer (10 mM Tris-HCl pH 7.6, 1 mM EDTA, 0.3 M NaCl). After the final wash, the plate is blotted on paper towels to remove excess wash buffer and then air-dried for 5 minutes. 60 μL of elution buffer (5 mM Tris-HCl pH 7.6), preheated to 70° C., is added to each well, the plate is incubated on a 90° C. hot plate for 5 minutes, and the eluate is then transferred to a fresh 96-well plate. Cells grown on 100 mm or other standard plates may be treated similarly, using appropriate volumes of all solutions.

Total RNA is isolated using an RNEASY 96™ kit and buffers can be purchased from Qiagen Inc. (Valencia, Calif. ) following the manufacturer's recommended procedures. Briefly, for cells grown on 96-well plates, growth medium is removed from the cells and each well is washed with 200 μL cold PBS. 150 μL Buffer RLT is added to each well and the plate vigorously agitated for 20 seconds. 150 μL of 70% ethanol is then added to each well and the contents mixed by pipetting three times up and down. The samples are then transferred to the RNEASY 96™ well plate attached to a QIA VAC™ mainifold fitted with a waste collection tray and attached to a vacuum source. Vacuum is applied for 1 minute. 500 μL of Buffer RW 1 is added to each well of the RNEASY 96™ plate and incubated for 15 minutes and the vacuum is again applied for 1 minute. An additional 500 μL of Buffer RW1 is added to each well of the RNEASY 96™ plate and the vacuum is applied for 2 minutes. 1 mL of Buffer RPE is then added to each well of the RNEASY 96™ plate and the vacuum applied for a period of 90 seconds. The Buffer RPE wash is then repeated and the vacuum is applied for an additional 3 minutes. The plate is then removed from the QIA VAC™ manifold and blotted dry on paper towels. The plate is then re-attached to the QIA VAC™ manifold fitted with a collection tube rack containing 1.2 mL collection tubes. RNA is then eluted by pipetting 140 μL of RNAse free water into each well, incubating 1 minute, and then applying the vacuum for 3 minutes.

The repetitive pipetting and elution steps may be automated using a QIAGEN Bio-Robot 9604 (Qiagen, Inc., Valencia Calif.). Essentially, after lysing of the cells on the culture plate, the plate is transferred to the robot deck where the pipetting, DNase treatment and elution steps are carried out.

In order that the invention disclosed herein may be more efficiently understood, examples are provided below. It should be understood that these examples are for illustrative purposes only and are not to be construed as limiting the invention in any manner. Throughout these examples, molecular reactions and other standard recombinant DNA techniques, were carried out according to methods described in Maniatis et al., Molecular Cloning—A Laboratory Manual, 2nd ed., Cold Spring Harbor Press (1989), using commercially available reagents, except where otherwise noted.

### EXAMPLES

### Example 1

### Representative Probe Compound and Target Nucleic Acid Polymer

A representative example of a target nucleic acid polymer is a micro RNA target sequence (mir-140), which comprises the nucleotide sequence AGUGGUUUUACCCUAUG GUAG (SEQ ID NO: 1). A representative probe compound of the invention comprises the nucleotide sequence R¹—CTATGGUCCACUACCAUAGGGUAAAACCACUGCTGGTTT TA-R² (SEQ ID NO:2). The bold nucleotides indicate nucleotides that are complementary to the target nucleic acid polymer. These nucleotides can also comprise chemical modifications such as, for example, 2′-O-methyl. Additional nucleobases that can comprise a 2′-O-methyl are underlined. As discussed above, one of R¹ and R² is a fluorescent reporter group and the other of R¹ and R² is a quenching agent.

In the absence of the target nucleic acid polymer, the probe compound can form the structure:

```
    C U A C C A U A G G G U A A A A C C A C U
A | | | | | | | | | | | | | | | G
    C C U G G T A T C -R¹ R²-A T T T T G G T C
```

wherein the fluorescent reporter group and the quenching agent are in close spatial proximity. In this instance, little no fluorescence emission is detected.

In the presence of the target nucleic acid polymer, the probe-target duplex can form the structure:

```
                                GAUGGUAUCCCAUUUUGGUGA
                                |||||||||||||||||||||
R¹-CTATGGUCCA - CUACCAUAGGGUAAAACCACU - GCTGGTTTTA
-R²
```

wherein the fluorescent reporter group and the quenching agent are not in close spatial proximity. In this instance, fluorescence emission can be detected.

### Example 2

### Representative Probe Compound

Another representative probe compound comprises the sequence UGGATTUCACU CAAATCCAGAGGCUAGCAGACCTAGCC (SEQ ID NO:3), which is related to TGGATT TCACTCAAATCCAGAGGCTAGCAGACCTAGCC (SEQ ID NO:4). A probe comprising this nucleotide sequence can be prepared using many different chemistries. One representative chemistry is shown below.

| Position | | | |
|---|---|---|---|
| Number | Heterocycle | Linker | Sugar |
| 1 | uracil | phosphate ester | 2'-O-methylribose |
| 2 | guanine | phosphate ester | 2'-deoxyribose |
| 3 | guanine | phosphate ester | 2'-deoxyribose |
| 4 | adenine | phosphate ester | 2'-deoxyribose |
| 5 | thymine | phosphate ester | 2'-deoxyribose |
| 6 | thymine | phosphate ester | 2'-deoxyribose |
| 7 | uracil | phosphate ester | 2'-O-methylribose |
| 8 | cytosine | phosphate ester | 2'-deoxyribose |
| 9 | adenine | phosphate ester | 2'-deoxyribose |
| 10 | cytosine | phosphate ester | 2'-deoxyribose |
| 11 | uracil | phosphate ester | 2'-O-methylribose |
| 12 | cytosine | phosphate ester | 2'-O-methylribose |
| 13 | adenine | phosphate ester | 2'-O-methylribose |
| 14 | adenine | phosphate ester | 2'-O-methylribose |
| 15 | adenine | phosphate ester | 2'-O-methylribose |
| 16 | thymine | phosphate ester | 2'-O-methylribose |
| 17 | cytosine | phosphate ester | 2'-O-methylribose |
| 18 | cytosine | phosphate ester | 2'-O-methylribose |
| 19 | adenine | phosphate ester | 2'-O-methylribose |
| 20 | guanine | phosphate ester | 2'-O-methylribose |
| 21 | adenine | phosphate ester | 2'-O-methylribose |
| 22 | guanine | phosphate ester | 2'-O-methylribose |
| 23 | guanine | phosphate ester | 2'-O-methylribose |
| 24 | cytosine | phosphate ester | 2'-O-methylribose |
| 25 | uracil | phosphate ester | 2'-O-methylribose |
| 26 | adenine | phosphate ester | 2'-O-methylribose |
| 27 | guanine | phosphate ester | 2'-O-methylribose |
| 28 | cytosine | phosphate ester | 2'-O-methylribose |
| 29 | adenine | phosphate ester | 2'-O-methylribose |
| 30 | guanine | phosphate ester | 2'-O-methylribose |
| 31 | adenine | phosphate ester | 2'-deoxyribose |
| 32 | cytosine | phosphate ester | 2'-deoxyribose |
| 33 | cytosine | phosphate ester | 2'-O-methylribose |
| 34 | thymine | phosphate ester | 2'-deoxyribose |
| 35 | adenine | phosphate ester | 2'-deoxyribose |
| 36 | guanine | phosphate ester | 2'-deoxyribose |
| 37 | cytosine | phosphate ester | 2'-deoxyribose |
| 38 | cytosine | no linker | 2'-O-methylribose |

For this compound, a 3′-Cy5 fluorescent reporter group and 5′-Iowa Black™ agent was used.

### Example 3

### Representative Probe Compound

Another representative probe compound comprises the sequence TGGATTTCACU CAAATCCAGAGGCUAGCAGACCTAGCC (SEQ ID NO:5), which is also related to SEQ ID NO: 4. A probe comprising this nucleotide sequence can be prepared using many different chemistries. One representative chemistry is shown below.

| Position | | | |
|---|---|---|---|
| Number | Heterocycle | Linker | Sugar |
| 1 | thymine | phosphate ester | 2'-deoxyribose |
| 2 | guanine | phosphate ester | 2'-deoxyribose |
| 3 | guanine | phosphate ester | 2'-deoxyribose |
| 4 | adenine | phosphate ester | 2'-deoxyribose |
| 5 | thymine | phosphate ester | 2'-deoxyribose |
| 6 | thymine | phosphate ester | 2'-deoxyribose |
| 7 | thymine | phosphate ester | 2'-deoxyribose |
| 8 | cytosine | phosphate ester | 2'-deoxyribose |
| 9 | adenine | phosphate ester | 2'-deoxyribose |
| 10 | cytosine | phosphate ester | 2'-deoxyribose |
| 11 | uracil | phosphate ester | 2'-O-methylribose |
| 12 | cytosine | phosphate ester | 2'-O-methylribose |
| 13 | adenine | phosphate ester | 2'-O-methylribose |
| 14 | adenine | phosphate ester | 2'-O-methylribose |
| 15 | adenine | phosphate ester | 2'-O-methylribose |
| 16 | thymine | phosphate ester | 2'-O-methylribose |
| 17 | cytosine | phosphate ester | 2'-O-methylribose |
| 18 | cytosine | phosphate ester | 2'-O-methylribose |
| 19 | adenine | phosphate ester | 2'-O-methylribose |
| 20 | guanine | phosphate ester | 2'-O-methylribose |
| 21 | adenine | phosphate ester | 2'-O-methylribose |
| 22 | guanine | phosphate ester | 2'-O-methylribose |
| 23 | guanine | phosphate ester | 2'-O-methylribose |
| 24 | cytosine | phosphate ester | 2'-O-methylribose |
| 25 | uracil | phosphate ester | 2'-O-methylribose |
| 26 | adenine | phosphate ester | 2'-O-methylribose |
| 27 | guanine | phosphate ester | 2'-O-methylribose |
| 28 | cytosine | phosphate ester | 2'-O-methylribose |
| 29 | adenine | phosphate ester | 2'-O-methylribose |
| 30 | guanine | phosphate ester | 2'-O-methylribose |
| 31 | adenine | phosphate ester | 2'-deoxyribose |
| 32 | cytosine | phosphate ester | 2'-deoxyribose |
| 33 | cytosine | phosphate ester | 2'-deoxyribose |
| 34 | thymine | phosphate ester | 2'-deoxyribose |
| 35 | adenine | phosphate ester | 2'-deoxyribose |
| 36 | guanine | phosphate ester | 2'-deoxyribose |
| 37 | cytosine | phosphate ester | 2'-deoxyribose |
| 38 | cytosine | no linker | 2'-deoxyribose |

For this compound, a 3′-Cy5 fluorescent reporter group and 5′-Iowa Black™ quenching agent was used.

### Example 4

### Representative Probe Compound

Another representative probe compound comprises the sequence UUUGUCUCUGG UCCUUACUU (SEQ ID NO:6), which is related to TTTGTCTCTGGTCCTTACTT (SEQ ID No:7) A probe comprising this nucleotide sequence can also be prepared using many different chemistries. Two different representative chemistries are shown below.

Chemistry 1

| Position | | | |
|---|---|---|---|
| Number | Heterocycle | Linker | Sugar |
| 1 | uracil | phosphate ester | ribose |
| 2 | uracil | phosphate ester | ribose |
| 3 | uracil | phosphate ester | ribose |
| 4 | guanine | phosphate ester | ribose |
| 5 | uracil | phosphate ester | ribose |
| 6 | cytosine | phosphate ester | ribose |
| 7 | uracil | phosphate ester | ribose |
| 8 | cytosine | phosphate ester | ribose |
| 9 | uracil | phosphate ester | ribose |
| 10 | guanine | phosphate ester | ribose |
| 11 | guanine | phosphate ester | ribose |
| 12 | uracil | phosphate ester | ribose |
| 13 | cytosine | phosphate ester | ribose |
| 14 | cytosine | phosphate ester | ribose |
| 15 | uracil | phosphate ester | ribose |
| 16 | uracil | phosphate ester | ribose |
| 17 | adenine | phosphate ester | ribose |
| 18 | cytosine | phosphate ester | ribose |
| 19 | uracil | phosphate ester | ribose |
| 20 | uracil | no linker | ribose |

For this compound, a 3′-Cy5 fluorescent reporter group was used. This probe targets an siRNA molecule for PTEN.

Chemistry 2

| Position | | | |
|---|---|---|---|
| Number | Heterocycle | Linker | Sugar |
| 1 | uracil | thioate ester | ribose |
| 2 | uracil | thioate ester | ribose |
| 3 | uracil | thioate ester | ribose |
| 4 | guanine | thioate ester | ribose |
| 5 | uracil | thioate ester | ribose |
| 6 | cytosine | thioate ester | ribose |
| 7 | uracil | thioate ester | ribose |
| 8 | cytosine | thioate ester | ribose |
| 9 | uracil | thioate ester | ribose |
| 10 | guanine | thioate ester | ribose |
| 11 | guanine | thioate ester | ribose |
| 12 | uracil | thioate ester | ribose |
| 13 | cytosine | thioate ester | ribose |
| 14 | cytosine | thioate ester | ribose |
| 15 | uracil | thioate ester | ribose |
| 16 | uracil | thioate ester | ribose |
| 17 | adenine | thioate ester | ribose |
| 18 | cytosine | thioate ester | ribose |
| 19 | uracil | thioate ester | ribose |
| 20 | uracil | no linker | ribose |

For this compound, a 3′-Cy5 fluorescent reporter group was used. This probe targets an siRNA molecule for PTEN.

### Example 5

### Representative Probe Compound

Another representative probe compound comprises the sequence AAGUAAGGACC AGAGACAAA (SEQ ID NO:8), which is related to AAGTAAGGACCAGAGACAAA (SEQ ID NO:9). A probe comprising this nucleotide sequence can also be prepared using many different chemistries. Two different representative chemistries are shown below.

Chemistry 1

| Position | | | |
|---|---|---|---|
| Number | Heterocycle | Linker | Sugar |
| 1 | adenine | phosphate ester | ribose |
| 2 | adenine | phosphate ester | ribose |
| 3 | guanine | phosphate ester | ribose |
| 4 | uracil | phosphate ester | ribose |
| 5 | adenine | phosphate ester | ribose |
| 6 | adenine | phosphate ester | ribose |
| 7 | guanine | phosphate ester | ribose |
| 8 | guanine | phosphate ester | ribose |
| 9 | adenine | phosphate ester | ribose |
| 10 | cytosine | phosphate ester | ribose |
| 11 | cytosine | phosphate ester | ribose |
| 12 | adenine | phosphate ester | ribose |
| 13 | guanine | phosphate ester | ribose |
| 14 | adenine | phosphate ester | ribose |
| 15 | guanine | phosphate ester | ribose |
| 16 | adenine | phosphate ester | ribose |
| 17 | cytosine | phosphate ester | ribose |
| 18 | adenine | phosphate ester | ribose |
| 19 | adenine | phosphate ester | ribose |
| 20 | adenine | no linker | ribose |

For this compound, a 5′-Iowa Black™ quenching agent was used. This probe targets an siRNA molecule for PTEN.

Chemistry 2

| Position | | | |
|---|---|---|---|
| Number | Heterocycle | Linker | Sugar |
| 1 | adenine | thioate ester | ribose |
| 2 | adenine | thioate ester | ribose |
| 3 | guanine | thioate ester | ribose |
| 4 | uracil | thioate ester | ribose |
| 5 | adenine | thioate ester | ribose |
| 6 | adenine | thioate ester | ribose |
| 7 | guanine | thioate ester | ribose |
| 8 | guanine | thioate ester | ribose |
| 9 | adenine | thioate ester | ribose |
| 10 | cytosine | thioate ester | ribose |
| 11 | cytosine | thioate ester | ribose |
| 12 | adenine | thioate ester | ribose |
| 13 | guanine | thioate ester | ribose |
| 14 | adenine | thioate ester | ribose |
| 15 | guanine | thioate ester | ribose |
| 16 | adenine | thioate ester | ribose |
| 17 | cytosine | thioate ester | ribose |
| 18 | adenine | thioate ester | ribose |
| 19 | adenine | thioate ester | ribose |
| 20 | adenine | no linker | ribose |

For this compound, a 5′-Iowa Black™ quenching agent was used. This probe targets an siRNA molecule for PTEN.

Various modifications of the invention, in addition to those described herein, will be apparent to those skilled in the art from the foregoing description. Such modifications are also intended to fall within the scope of the appended claims. Each reference, patent, and international publication cited or referred to in the present application is incorporated herein by reference in its entirety.

1. A compound comprising the structure:
R¹—Uₙ-Lₖ-Wₐ—Xᵣ—Yₚ-Eₘ-Z_{b}-R²
wherein:
one of R¹ and R² is a fluorescent reporter group and the other of R¹ and R² is a quenching agent, or both R¹ and R² are fluorescent reporter groups;
each U and each W are nucleobases, wherein at least one U nucleobase is complementary to a corresponding W nucleobase;
each Y and each Z are nucleobases, wherein at least one Y nucleobase is complementary to a corresponding Z nucleobase;
each L and each E is a nucleobase that, when a plurality of U and a plurality of W nucleobases are basepaired and a plurality of Y and a plurality of Z nucleobases are basepaired, forms a loop;
each X is a nucleobase that, when a plurality of U and a plurality of W nucleobases are basepaired and a plurality of Y and a plurality of Z nucleobases are basepaired, is not basepaired to any nucleobase of the compound;
n, a, p, and b are, independently, 4 to 10;
k and m are, independently, 1 to 10; and
r is 0 to 4.

2. A compound of claim 1 wherein one of R¹ and R² is a fluorescent reporter group and the other of R¹ and R² is a quenching agent.

3. A compound of claim 1 wherein both R¹ and R² are fluorescent reporter groups.

4. A compound of claim 1 wherein the fluorescent reporter group is a fluorescent base or a fluorophore.

5. A compound of claim 4 wherein the fluorophore is tetramethylrhodamine, fluorescein, Cy3, Cy3.5, Cy5, Cy5.5, or Texas Red.

6. A compound of claim 1 wherein the quenching agent is an arylazo compound.

7. A compound of claim 6 wherein the arylazo compound is 4-(4′-dimethylaminophenylazo)benzoic acid, a Black Hole Quencher™, or Iowa Black™.

8. A compound of claim 1 wherein at least one W, X, or Y nucleobase comprises a chemical modification.

9. A compound of claim 8 wherein the chemical modification is 2′-O-methyl, 2′-O-methoxyethyl, or 2′F.

10. A compound of claim 1 wherein at least one U or Z nucleobase comprises a chemical modification.

11. A compound of claim 10 wherein the chemical modification is 2′-deoxy or 2′-arafluoro.

12. A compound of claim 1 comprising less than five contiguous deoxyribonucleotides.

13. A compound of claim 1 wherein n, a, p, and b are, independently, 5 to 7; k and m are, independently, 3 to 8; and r is 1 or 2.

14. A compound of claim 1 comprising SEQ ID NO:2, SEQ ID NO:3, SEQ ID NO:5, SEQ ID NO:6, or SEQ ID NO:8.

15. A method of detecting a target nucleic acid polymer comprising detecting the presence of a fluorescent signal produced by a compound of claim 1 when contacted with the target nucleic acid polymer.

16. A method of claim 15 wherein the target nucleic acid polymer is an RNA molecule.

17. A method of claim 16 wherein the RNA molecule is a small interfering RNA, mRNA, antisense RNA, or micro RNA.

18. A method of claim 15 wherein the detection of the fluorescent signal is performed by flow cytometry or microscopy.

19. A method of claim 15 wherein the compound of claim 1 and the target nucleic acid polymer are present within a cell.

20. A method of claim 19 wherein the compound of claim 1 is transfected or electroporated into the cell.

21. A method of claim 15 wherein the target nucleic acid polymer is present within a cell supernatant after lysis of the cell, and the fluorescent signal detected using laser-induced fluorescence excitation and detection.

22. A method of claim 15 wherein the target nucleic acid polymer is present within a fixed tissue section.

23. A method of claim 15 further comprising quantifying the amount of the target nucleic acid polymer.

24. A method of claim 23 wherein the quantifying is performed by comparing the level of the fluorescent signal obtained from contacting the compound of claim 1 to the target nucleic acid polymer to the level of fluorescent signal obtained from a positive control.

25. A kit comprising a compound of claim 1.
